(19) United States
(12) Patent Application Publication
(10) Pub. No.: US 2012/0027170 A1
(43) Pub. Date: Feb. 2, 2012

(54) RADIOLOGICAL IMAGE CAPTURING AND DISPLAYING METHOD AND APPARATUS

(75) Inventor: Hiroki NAKAYAMA, Ashigarakami-gun (JP)

(73) Assignee: FUJIFILM CORPORATION, Tokyo (JP)

(21) Appl. No.: 13/190,912

(22) Filed: Jul. 26, 2011

(30) Foreign Application Priority Data
Jul. 29, 2010 (JP) 2010-169945

(51) Int. Cl. A61B 6/02

(52) U.S. Cl. 378/42

(57) ABSTRACT

Even when one side of a radiological image detector is inclined with respect to a straight line linking two focal positions of radiation, an appropriate stereoscopic image is displayed. The amount d of shift between two radiological images in a direction perpendicular to a parallax direction is acquired and a stereoscopic image is displayed using the two radiological images which are relatively shifted in the direction perpendicular to the parallax direction so that the acquired amount d of shift is reduced.

### BACKGROUND OF THE INVENTION

1. Field of the Invention

The present invention relates to a radiological image capturing and displaying method and apparatus that captures a subject in different radiographing directions to acquire radiological images from the different radiographing directions and displays a stereoscopic image using the radiological images.

2. Description of the Related Art

A technique is known which combines a plurality of images and displays the combined image, thereby enabling stereoscopically viewing using parallax. The image that can be stereoscopically viewed (hereinafter, referred to as a stereoscopic image or a stereo image) is generated based on a plurality of images which are acquired by capturing the same subject from different directions and have parallax therebetween.

Moreover, such way of generating stereoscopic image is utilized not only in the field of digital cameras and televisions but also in the field of capturing a stereoscopic radiological image. That is, a test subject is irradiated with radiation from different directions, and then the radiation passing through the test subject is detected by a radiological image detector to acquire plural radiological images having parallax, and a stereoscopic image is generated based on the radiological images. By generating a stereoscopic image in this way, a radiological image with a sense of depth can be observed thereby enabling the observation of a radiological image more suitable for diagnosis.

### SUMMARY OF THE INVENTION

In the apparatus that captures the stereo image, one side of the radiological image detector needs to be parallel to a straight line connecting the two focal positions of a radiation source that radiates radiation in different directions.

However, for example, as shown in FIG. 6, when the side of a radiological image detector 15 is inclined with respect to a straight line connecting a focal position S1 and a focal position S2, there is a shift in the Y direction between a radiological image which is captured by the radiation from the focal position S1 and a radiological image which is captured by the radiation from the focal position S2.

That is, as shown in FIGS. 7A and 7B, there is a shift between a subject image P1 and a subject image P2 indicating a subject OB in the Y direction.

As such, when there is a shift between two subject images P1 and P2 forming a stereo image in the Y direction and the observer observes the stereo image displayed on a monitor 3, the burden on the observer to recognize the stereo image increases, which results in an increase in the fatigue of the observer.

JP2004-105568A discloses a technique that compensates the distortion of an image due to a change in the distance between a radiation source and a radiological image detector when the radiological image detector is moved and captures an image. However, JP-A-2004-105568 does not relate to the capture of a stereo image and does not disclose the above-mentioned problems in the capture of the stereo image and a solution for the problems.

JP-S59-40840A (JP19814-40840A) discloses an imaging apparatus that captures a stereo image. However, JP-S59-40840A (JP1984-40840A) does not disclose the above-mentioned problems in the capture of the stereo image and a solution for the problems.

The present invention has been made in view of the above-mentioned problems and an object of the present invention is to provide a radiological image capturing and displaying method and apparatus capable of displaying an appropriate stereo image even when one side of a radiological image detector is inclined with respect to a straight line linking two focal positions of radiation.

According to an aspect of the present invention , there is provided a radiological image capturing and displaying method that radiates radiation emitted from two focal positions to a subject in two different radiographing directions respectively to acquire two radiological images in the two different radiographing directions and displays a stereo image using the two acquired radiological images. The radiological image capturing and displaying method includes: acquiring the shift amount between the two radiological images in a direction perpendicular to a parallax direction; and displaying the stereoscopic image using the two radiological images which are relatively shifted in the direction perpendicular to the parallax direction as to reduce the acquired shift amount.

According to another aspect of the present invention, there is provided a radiological image capturing and displaying apparatus including: a radiological image acquiring unit that radiates radiation from two focal positions to a subject in two different radiographing directions to acquire two radiological images in the two different radiographing directions; a display unit that displays a stereoscopic image using the two radiological images acquired by the radiological image acquiring unit; and a shift amount acquiring unit that acquires the shift amount between the two radiological images in a direction perpendicular to a parallax direction. The display unit displays the stereoscopic image using the two radiological images which are relatively shifted in the direction perpendicular to the parallax direction as to reduce the shift amount acquired by the shift amount acquiring unit.

In the radiological image capturing and displaying apparatus according to the above-mentioned aspect, the shift amount acquiring unit may acquire the shift amount between marker images which appear in the two radiological images by radiographing reference marker in the two radiographing directions.

The radiological images including the marker images may be acquired before the subject is captured.

The reference marker may be provided on a radiography platform on which the subject is placed.

The radiological image capturing and displaying apparatus according to the above-mentioned aspect may further include a compression plate that compresses the subject. The subject may be a breast, and the reference marker may be placed on the compression plate.

The radiological image capturing and displaying apparatus according to the above-mentioned aspect may further include a radiation source that moves the two focal positions and radiates the radiation. The reference marker may be placed on a housing of the radiation source.

The shift amount acquiring unit may acquire the shift amount based on the specific portion of the subject in each of the radiological images.

The specific portion may be the outline of the subject.

The subject may be the breast and the specific portion may be the nipple of the breast.

The radiological image capturing and displaying apparatus according to the above-mentioned aspect may further include a radiological image detector that has a rectangular shape and detects the radiological image. The shift amount acquiring unit may acquire the shift amount based on the inclination of a side of the radiological image detector with respect to a straight line connecting the two focal positions.

The radiological image capturing and displaying apparatus according to the above-mentioned aspect may further include a radiological image compensating unit that performs a shift process on the two radiological images in the direction perpendicular to the parallax direction based on the shift amount acquired by the shift amount acquiring unit.

The radiological image capturing and displaying apparatus according to the above-mentioned aspect may further include a display control unit that controls the display unit to display the two radiological images shifted ( [symbol] shifted [symbol] which are [symbol] [symbol] ) in the direction perpendicular to the parallax direction based on the shift amount acquired by the shift amount acquiring unit.

The radiological image capturing and displaying apparatus according to the above-mentioned aspect may further include a cassette detecting unit that detects the insertion of a removable cassette. The radiological image detector may be provided in the removable cassette. When the cassette detecting unit detects the insertion of the cassette, the shift amount acquiring unit may automatically acquire the shift amount, or information prompting the acquisition of the shift amount may be notified.

According to the stereoscopic image capturing and displaying method and apparatus of the present invention, the shift amount between two radiological images in a direction perpendicular to the parallax direction is acquired, and a stereoscopic image is displayed using the two radiological images which are relatively shifted in the direction perpendicular to the parallax direction so that the acquired shift amount is reduced. Therefore, even when one side of the radiological image detector is inclined with respect to a straight line linking two focal positions of radiation, it is possible to display an appropriate stereoscopic image.

### BRIEF DESCRIPTION OF THE DRAWINGS

FIG. 1 is a diagram schematically illustrating the structure of a breast image capturing and displaying system using a radiological image capturing and displaying apparatus according to an embodiment of the present invention.

FIG. 2 is a diagram illustrating an arm unit of the breast image capturing and displaying system shown in FIG. 1, as viewed from the right direction of FIG. 1.

FIG. 3 is a block diagram schematically illustrating the internal structure of a computer of the breast image capturing and displaying system shown in FIG. 1.

FIG. 4 is a diagram illustrating a marker member provided on a radiography platform.

FIG. 5 is a flowchart illustrating the operation of the breast image capturing and displaying system using the radiological image capturing and displaying apparatus according to the embodiment of the present invention.

FIG. 6 is a diagram illustrating a case in which a radiological image detector is provided so that one side thereof is inclined with respect to a straight line linking two focal positions of radiation.

FIGS. 7A and 7B are diagrams illustrating an example of a first radiological image P1 which is captured by the emission of radiation from one of the focal positions to a subject and a second radiological image P2 which is captured by the emission of radiation from the other focal position to the subject in the arrangement position of the radiological image detector shown in FIG. 6.

FIG. 8 is a diagram illustrating the shift of the moving direction of the focal position of radiation from one side of the radiological image detector.

FIGS. 9A and 9B are diagrams illustrating an example of the first radiological image P1 which is captured by the emission of radiation from one of the focal positions to the subject and the second radiological image P2 which is captured by the emission of radiation from the other focal position to the subject in the relationship between the moving direction of the focal position and the arrangement position of the radiological image detector shown in FIG. 8.

FIG. 10 is a diagram illustrating an example in which the marker member is provided on a compression plate.

FIG. 11 is a diagram illustrating an example in which the marker member is provided on a housing of a radiation source.

### DESCRIPTION OF THE PREFERRED EMBODIMENTS

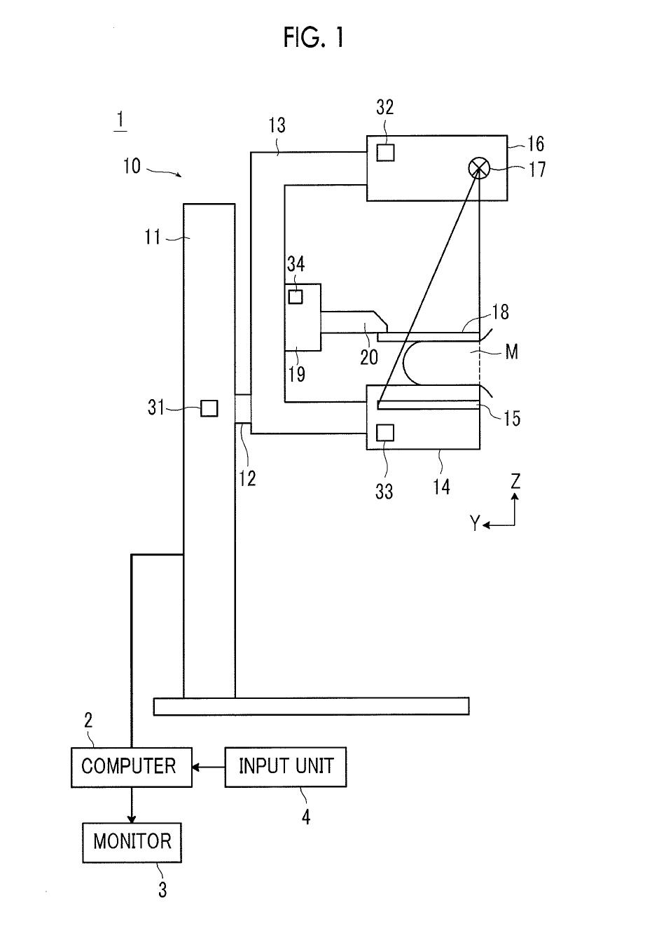

Hereinafter, a breast image capturing and displaying system using a radiological image capturing and displaying apparatus according to an embodiment of the present invention will be described with reference to the accompanying drawings. FIG. 1 is a diagram schematically illustrating the overall structure of the breast image capturing and displaying system according to this embodiment.

As shown in FIG. 1, a breast image capturing and displaying system 1 according to this embodiment includes a breast imaging apparatus 10, a computer 2 that is connected to the breast imaging apparatus 10, and a monitor 3 and an input unit 4 that are connected to the computer 2.

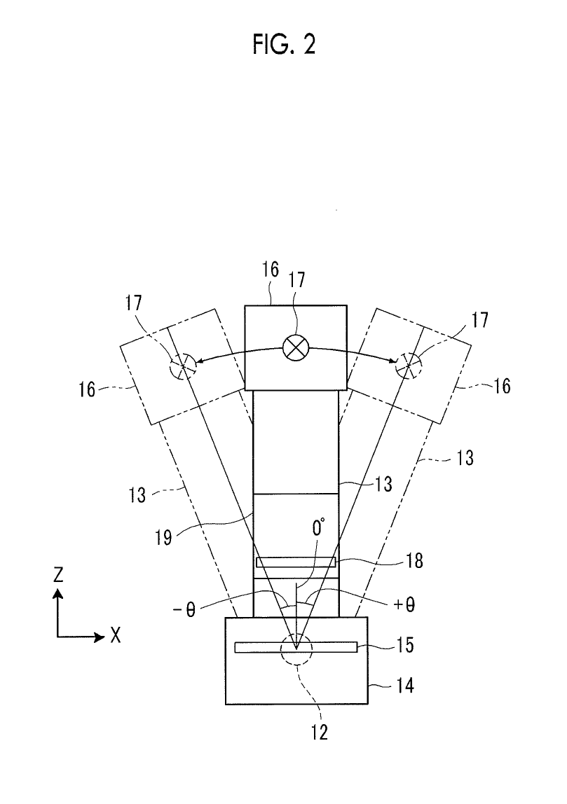

As shown in FIG. 1, the breast imaging apparatus 10 includes a base 11, a rotating shaft 12 that is movable in the vertical direction (Z direction) relative to the base 11 and is rotatable, and an arm unit 13 that is connected to the base 11 by the rotating shaft 12. FIG. 2 shows the arm unit 13, as viewed from the right direction of FIG. 1.

The arm unit 13 has a C-shape and includes one end to which a radiography platform 14 is attached and the other end to which a radiation radiating unit 16 is attached so as to face the radiography platform 14. The rotation and vertical movement of the arm unit 13 are controlled by an arm controller 31 that is incorporated into the base 11.

The radiography platform 14 includes a radiological image detector 15, such as a flat panel detector, and a detector controller 33 that controls the reading of a charge signal from the radiological image detector 15. In addition, the radiography platform 14 includes, for example, a circuit board provided with a charge amplifier that converts the charge signal read from the radiological image detector 15 into a voltage signal, a correlated double sampling circuit that samples the voltage signal output from the charge amplifier, and an A/D converter that converts the voltage signal into a digital signal.

The radiography platform 14 is configured so as to be rotatable with respect to the arm unit 13. Therefore, even when the arm unit 13 is rotated with respect to the base 11, the direction of the radiography platform 14 can be fixed with respect to the base 11.

The radiological image detector 15 can repeatedly record and read the radiological image and may be a so-called direct radiological image detector that directly receives radiation and generates charge or a so-called indirect radiological image detector that converts radiation into visible light and then converts the visible light into a charge signal. As a method of reading a radiological image signal, it is preferable to use a so-called TFT reading method of turning on or off a TFT (thin film transistor) switch to read the radiological image signal or a so-called optical reading method of radiating reading light to read the radiological image signal. However, the reading method is not limited thereto, but other methods may be used.

The radiation radiating unit 16 includes a radiation source 17 and a radiation source controller 32. The radiation source controller 32 controls the time when radiation is radiated from the radiation source 17 and the radiation generation conditions (for example, a tube current, time, and a tube current-time product) of the radiation source 17.

In addition, a compression plate 18 that is provided above the radiography platform 14 and compresses the breast, a supporting portion 20 that supports the compression plate 18, and a moving mechanism 19 that moves the supporting portion 20 in the vertical direction (Z direction) are provided at the center of the arm unit 13. The position and compression pressure of the compression plate 18 are controlled by a compression plate controller 34.

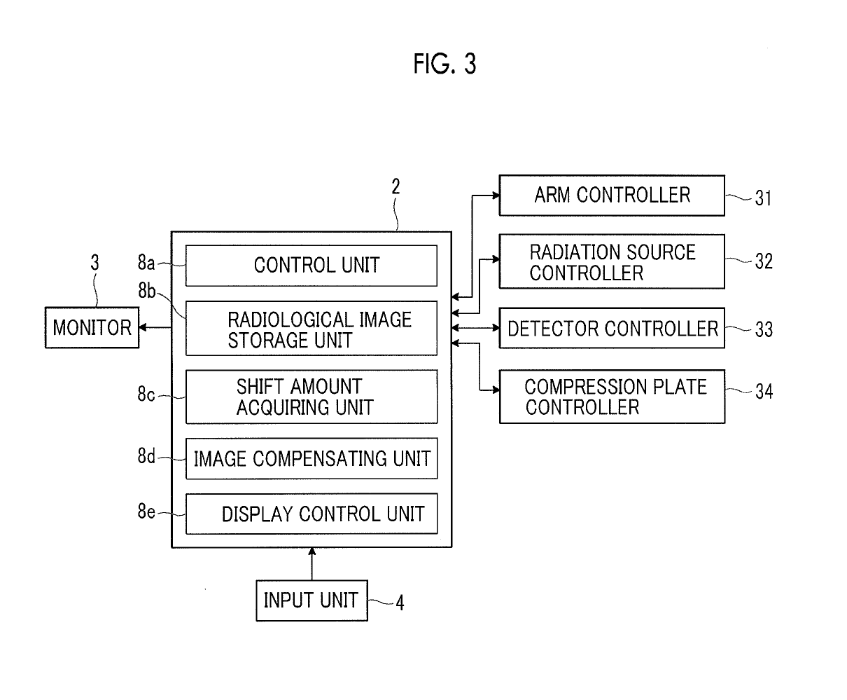

The computer 2 includes, for example, a central processing unit (CPU) and a storage device, such as a semiconductor memory, a hard disk, or an SSD. A control unit 8a, a radiological image storage unit 8b, a shift amount acquiring unit 8c, an image correcting unit 8d, and a display control unit 8e shown in FIG. 3 are formed by these hardware components.

The control unit 8a outputs predetermined control signals to various kinds of controllers 31 to 35 to control the entire system. A detailed control method will be described below.

The radiological image storage unit 8b stores the radiological image signals acquired by the radiological image detector 15 in advance.

The shift amount acquiring unit 8c acquires the shift amount between two radiological images forming a stereo image in a direction perpendicular to the parallax direction.

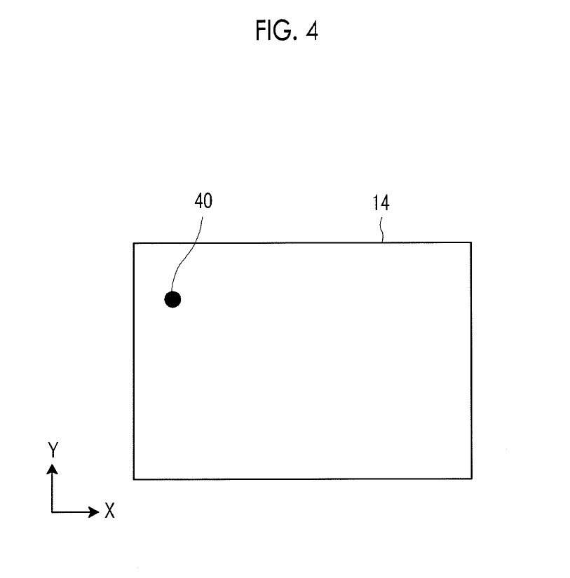

A marker member 40 is provided in the radiography platform 14 according to this embodiment, as shown in FIG. 4. The shift amount acquiring unit 8c acquires the shift amount between marker images appearing on the two radiological images by the marker member 40 as the shift amount between the two radiological images. In this embodiment, the marker member 40 is a radiation absorption member, such as lead, and is provided at the corner of the radiography platform 14 so that the marker image does not overlap the breast image. The shift amount acquiring operation of the shift amount acquiring unit 8c will be described in detail below.

The image correcting unit 8d performs a shift process of relatively shifting two radiological image signals so that the shift amount acquired by the shift amount acquiring unit 8c is reduced. Performing the shift process so that the shift amount is reduced means performing the shift process so that the shift amount is zero.

The display control unit 8e displays the stereo image of the breast on the monitor 3 based on the two radiological image signals shifted by the image correcting unit 8d.

The input unit 4 is, for example, a keyboard or a pointing device, such as a mouse, and receives imaging conditions or an imaging start instruction input from a photographer.

The monitor 3 is configured so that it can display a stereo image using two radiological image signals output from the computer 2 when the stereo image is captured. As a structure that displays the stereo image, for example, the following structure may be used in which two radiological images are respectively displayed on two screens based on two radiological image signals and, for example, a half mirror or a polarization glass is used so that one of the two radiological images is incident on the right eye of the observer and the other radiological image is incident on the left eye of the observer, thereby displaying a stereo image. Alternatively, for example, the following structure may be used: a structure in which two radiological images are displayed so as to overlap each other with a positional shift corresponding to a predetermined amount of parallax therebetween and a polarization glass is used to generate a stereo image so that the observer can view the stereo image; or a structure, such as a parallax barrier type or a lenticular type, in which two radiological images are displayed on a 3D liquid crystal display that can three-dimensionally display the radiological images, thereby generating a stereo image.

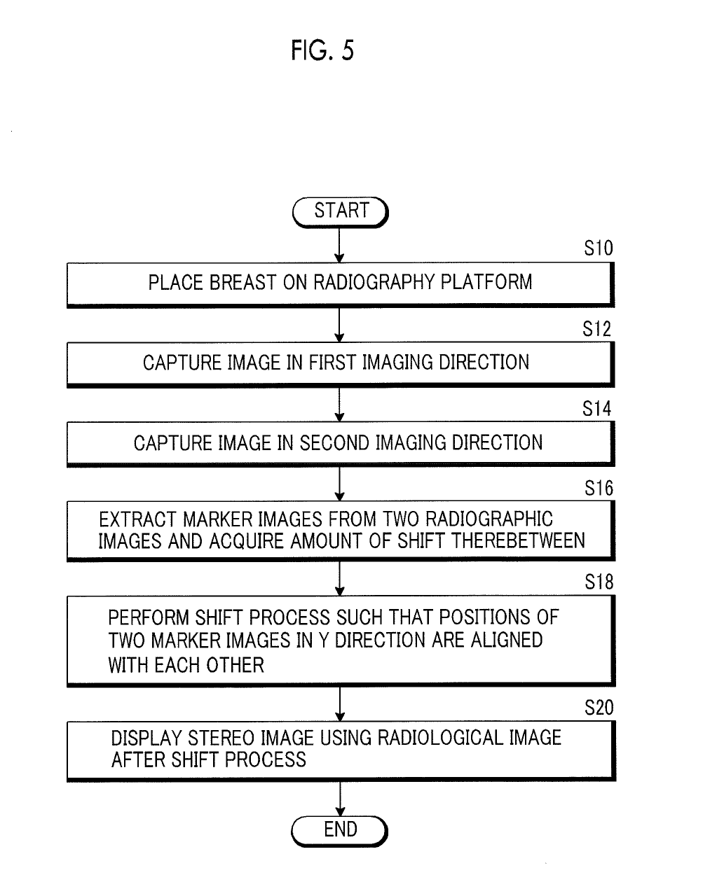

Next, the operation of the breast image capturing and displaying system according to this embodiment will be described with reference to the flowchart shown in FIG. 5.

First, the breast M of the patient is placed on the radiography platform 14 and the compression plate 18 compresses the breast M with a predetermined pressure (S10).

Then, the input unit 4 sequentially receives various kinds of imaging conditions and an image start instruction from the photographer.

When the input unit 4 receives the imaging start instruction, the stereo image of the breast M is captured (S12). Specifically, first, the control unit 8a reads the angle θ of convergence for capturing a predetermined stereo image and outputs the information of the read angle θ of convergence to the arm controller 31. In this embodiment, in this case, it is assumed that θ=±2° is stored as the information of the angle θ of convergence in advance, but the present invention is not limited thereto. The photographer may use the input unit 4 to set an arbitrary angle of convergence.

The arm controller 31 receives the information of the angle θ of convergence output from the control unit 8a. Then, the arm controller 31 outputs a control signal based on the information of the angle θ of convergence so that the arm unit 13 rotates +θ° with respect to the direction vertical to the radiography platform 14, as shown in FIG. 2. That is, in this embodiment, the arm controller 31 outputs a control signal so that the arm unit 13 rotates +2° with respect to the direction vertical to the radiography platform 14.

The arm unit 13 rotates +2° in response to the control signal output from the arm controller 31. Then, the control unit 8a outputs control signals to the radiation source controller 32 and the detector controller 33 so as to perform the emission of radiation and the reading of the radiological image signal, respectively. In response to the control signals, the radiation source 17 radiates radiation, the radiological image detector 15 detects the radiological image of the breast captured in a +2° direction, and the detector controller 33 reads the radiological image signal. Then, predetermined signal processing is performed on the radiological image signal, and the radiological image signal is stored in the radiological image storage unit 8b of the computer 2.

Then, as shown in FIG. 2, the arm controller 31 returns the arm unit to the initial position once and outputs a control signal so that the arm unit rotates −0° with respect to the direction vertical to the radiography platform 14 (S14). That is, in this embodiment, the arm controller 31 outputs a control signal so that the arm unit 13 rotates −2° with respect to the direction vertical to the radiography platform 14.

Then, the arm unit 13 rotates −2° in response to the control signal output from the arm controller 31. Then, the control unit 8a outputs control signals to the radiation source controller 32 and the detector controller 33 so as to perform the emission of radiation and the reading of the radiological image, respectively. In response to the control signals, the radiation source 17 radiates radiation, the radiological image detector 15 detects the radiological image of the breast captured in a −2° direction, and the detector controller 33 reads the radiological image signal. Then, predetermined signal processing is performed on the radiological image signal and the radiological image signal is stored in the radiological image storage unit 8b of the computer 2.

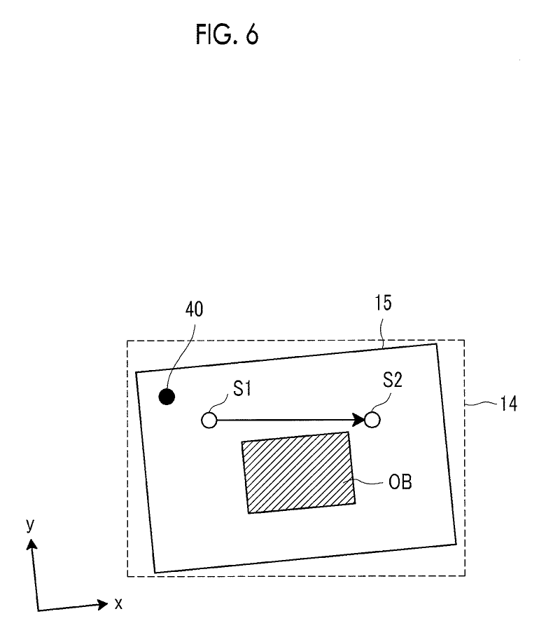

When, the radiological image detector 15 is arranged so that the side thereof is inclined with respect to a straight line linking the focal position S1 of the radiation source 17 when an image is captured in the +θ° direction and the focal position S2 of the radiation source when an image is captured in the −θ° direction, as shown in FIG. 6, the radiological image captured by radiation radiated from the focal position S1 deviates from the radiological image captured by radiation radiated from the focal position S2 in a direction perpendicular to the parallax direction.

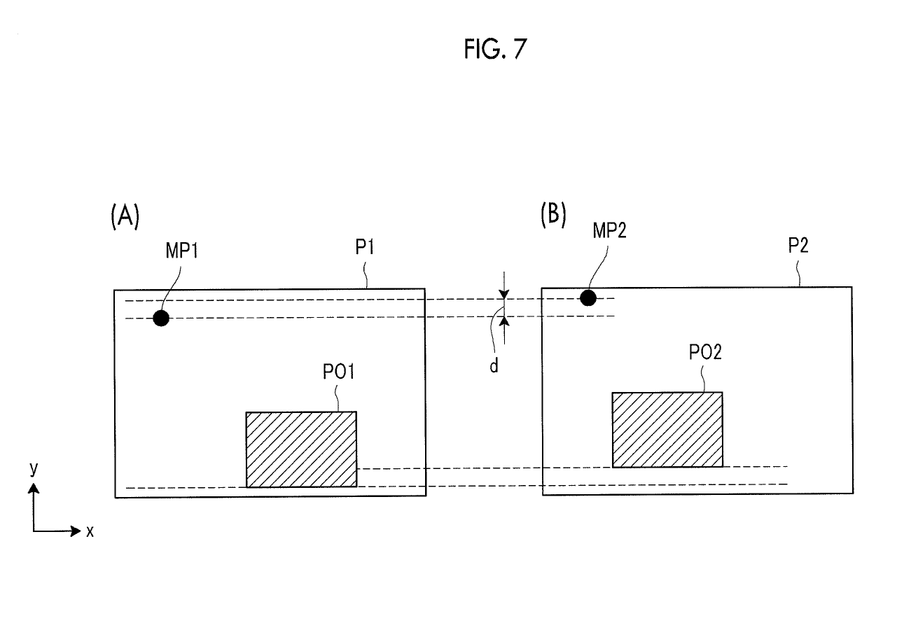

For ease of explanation, FIG. 6 shows a subject OB which schematically shows the breast M, FIG. 7A shows a first radiological image P1 which is captured by the emission of radiation from the focal position S1 to the subject OB, and FIG. 7B shows a second radiological image P2 which is captured by the emission of radiation from the focal position S2 to the subject OB.

When the radiological image detector 15 is provided so as to be inclined as shown in FIG. 6, a shift between the subject image P1 and the subject image P2 indicating the subject OB occurs in a direction perpendicular to the parallax direction, that is, the y direction shown in FIGS. 7A and 7B, as shown in FIGS. 7A and 7B. The x-y directions shown in FIG. 6 and FIGS. 7A and 7B are the coordinate axes of the radiological image detected by the radiological image detector 15.

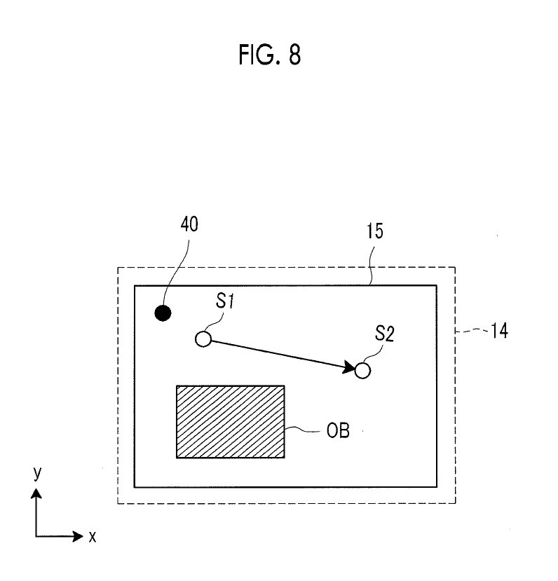

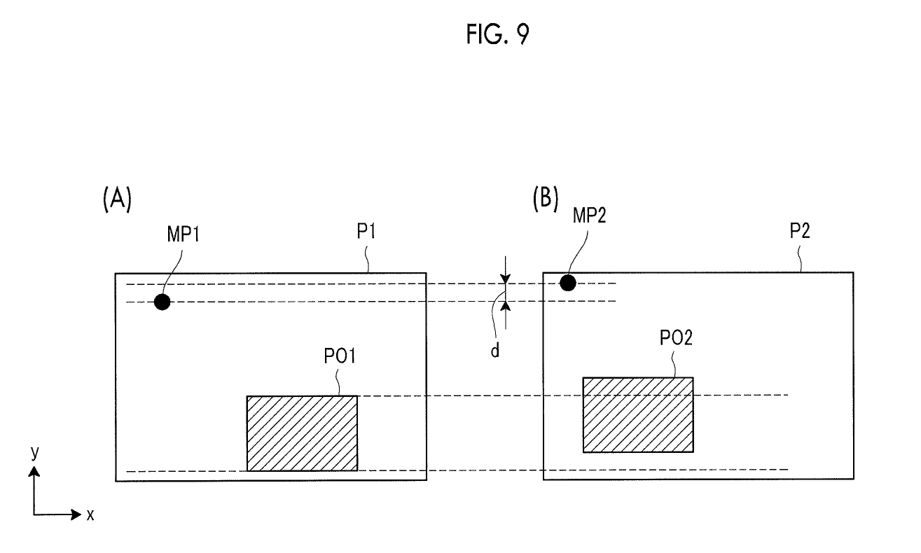

When the radiological image detector 15 is normally provided, but the straight line linking the two focal positions is inclined with respect to the side of the radiological image detector 15 due to the shift of the moving direction of the focal position of the radiation source 17 as shown in FIG. 8, the shift between the subject image P1 and the subject image P2 indicating the subject OB also occurs in the direction perpendicular to the parallax direction, that is, the y direction shown in FIGS. 9A and 9B, as shown in FIGS. 9A and 9B. The x-y directions shown in FIG. 8 and FIGS. 9A and 9B are also the coordinate axes of the radiological image detected by the radiological image detector 15.

As such, when the shift between the two subject images P1 and P2 forming the stereo image occurs in the y direction and the observer observes the stereo image displayed on the monitor 3, the burden on the observer to recognize the stereo image increases, which results in an increase in the fatigue of the observer.

Therefore, in the breast image capturing and displaying system according to this embodiment, the shift amount between the subject image P1 and the subject image P2 in the y direction is acquired and a process of shifting the two radiological images in the y direction is performed based on the shift amount.

Specifically, as described above, the two acquired radiological image signals are read from the radiological image storage unit 8b and are then input to the shift amount acquiring unit 8c. The shift amount acquiring unit 8c extracts the marker images from the input two radiological image signals and acquires the shift amount between the marker images in the y direction (S16). For example, when a marker image MP1 appears in one of the radiological images as shown in FIG. 7A or FIG. 9A and a marker image MP2 appears in the other radiological image as shown in FIG. 7B or FIG. 9B, the amount d of shift between the marker image MP1 and the marker image MP2 in the y direction is acquired.

Then, the amount d of shift acquired by the shift amount acquiring unit 8c is output to the image correcting unit 8d. The image correcting unit 8d performs a shift process on the two radiological image signals in the y direction based on the shift amount so that the amount d of shift is zero (S18). In this case, the shift process may be performed to shift only one radiological image signal in the y direction by the amount d of shift or the shift process may be performed to shift each of the two radiological image signals in the y direction by half the amount d of shift (d/2).

Then, the image correcting unit 8d outputs the two radiological image signals subjected to the shift process to the display control unit 8e and the display control unit 8e displays the stereo image of the breast on the monitor 3 based on the input two radiological image signals (S20).

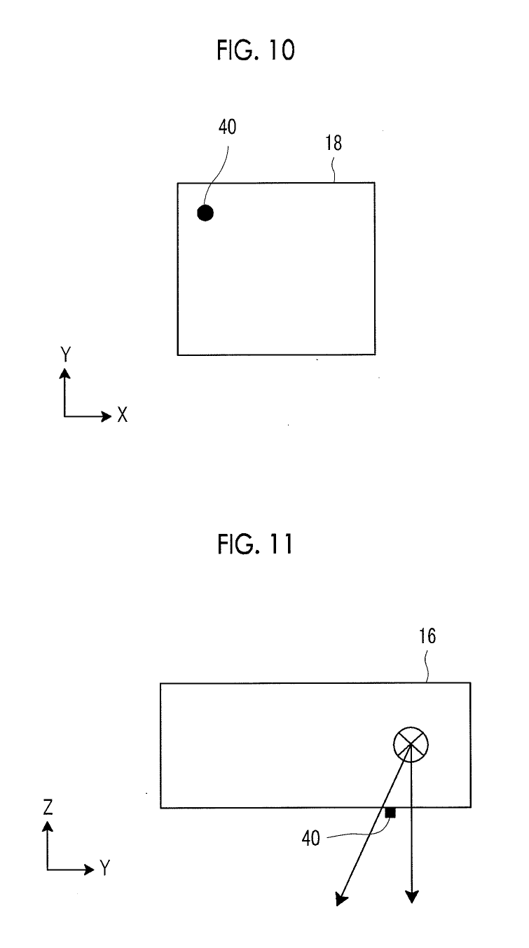

In the above-described embodiment, the marker member 40 is provided on the radiography platform 14, but the present invention is not limited thereto. For example, as shown in FIG. 10, the marker member 40 may be provided at the corner of the compression plate 18. In addition, as shown in FIG. 11, the marker member 40 may be provided on a housing of the radiation radiating unit 16 provided with the radiation source 17.

The marker image may be captured at the same time as the subject is captured as in the above-described embodiment, or it may be captured in advance by pre-exposure before the subject is captured.

In the above-described embodiment, the marker image of the marker member 40 is used to acquire the shift amount, but the marker image is not necessarily used. For example, the shift amount may be acquired based on the image of a specific portion in each radiological image.

For example, when the image of the specific portion is the radiological image of the breast, the skin line (outline) of the breast may be detected and the shift amount of the skin line in the y direction may be acquired. As a method of detecting the skin line, for example, a known edge detecting method may be used.

In addition, the position of a nipple in the radiological image of each breast may be detected and the shift amount at the position of the nipple in the y direction may be acquired. As a method of detecting the position of the nipple of the breast, for example, the following methods may be used: a method which detects the skin line of the breast and detects the position of the top of the skin line as the position of the nipple; and a method which detects a mammary gland pattern and detects the position where the density of the mammary gland pattern is the highest as the position of the nipple. In addition, other known methods may be used to detect the position of the nipple.

In addition, the inclination of the side of the rectangular radiological image detector 15 with respect to the straight line linking two focal positions may be directly detected and the shift amount of the breast image in the y direction may be acquired based on the inclination. For example, a laser displacement meter may be used to detect the inclination. In addition, the inclination may be measured in advance, the inclination may be input by the input unit 4, and the shift amount acquiring unit 8c may calculate the shift amount based on the input inclination.

In the above-described embodiment, the image correcting unit 8d performs the shift process on the radiological image signal based on the shift amount acquired by the shift amount acquiring unit 8c. However, instead of performing the shift process, the display control unit 8e may generate a display control signal based on the shift amount and two radiological image signals so that the two radiological images are displayed on the monitor 3 so as to be shifted by the shift amount, and output the display control signal to the monitor 3.

In addition, the radiological image detector 15 may be provided in a cassette that is removable from the radiography platform 14. In this case, when the cassette is inserted, the radiological image detector 15 can be inclined and the apparatus can detect the insertion of the cassette. When the insertion of the cassette is detected, the shift amount may be automatically acquired or information prompting the acquisition of the shift amount may be displayed. For example, the marker image may be automatically captured or information prompting the capture of the marker image may be displayed.

In the above description, the stereo image display apparatus according to an embodiment of the present invention is applied to the breast image capturing and displaying system, but the subject of the present invention is not limited to the breast. For example, the present invention can be applied to a radiological image capturing and displaying system that captures an image of the chest or the head.

What is claimed is:

1. A radiologocal image capturing and displaying method that radiates radiation emitted from two focal positions to a subject in two different radiographing directions respectively to acquire two radiological images in the two different radiographing directions, and displays a stereoscopic image using the two acquired radiological images, wherein the method comprising the steps of:
acquiring a shift amount between the two radiologocal images in a direction perpendicular to a parallax direction; and
displaying the stereoscopic image using the two radiological images which are relatively shifted in the direction perpendicular to the parallax direction as to reduce the acquired shift amount.

2. A radiological images capturing and displaying apparatus comprising:
a radiological images acquiring unit that radiates radiation emitted from two focal positions to a subject in two different radiographing directions respectively to acquire two radiological images in the two different radiographing directions;
a display unit that displays a stereoscopic image using the two radiological images acquired by the radiological images acquiring unit; and
a shift amount acquiring unit that acquires the shift amount between the two radiological images in a direction perpendicular to a parallax direction,
wherein the display unit displays the stereoscopic image using the two radiological images which are relatively shifted in the direction perpendicular to the parallax direction as to reduce the shift amount acquired by the shift amount acquiring unit.

3. The radiological image capturing and displaying apparatus according to claim 2,
wherein the shift amount acquiring unit acquires the shift amount between marker images which appear in the two radiological images by radiographing a reference marker in the two radiographing directions.

4. The radiological image capturing and displaying apparatus according to claim 3,
wherein the radiological images including the marker images are acquired before the subject is radiographed.

5. The radiological image capturing and displaying apparatus according to claim 3,
wherein the reference marker is provided on a radiography platform on which the subject is placed.

6. The radiological image capturing and displaying apparatus according to claim 3, further comprising:
a compression plate that compresses the subject,
wherein the subject is the breast, and
the reference marker is placed on the compression plate.

7. The radiological image capturing and displaying apparatus according to claim 3, further comprising:
a radiation source that moves the two focal positions and radiates the radiation,
wherein the reference marker is placed on a housing of the radiation source.

8. The radiological image capturing and displaying apparatus according to claim 4, further comprising:
a radiation source that moves the two focal positions and radiates the radiation,
wherein the reference marker is placed on a housing of the radiation source.

9. The radiological image capturing and displaying apparatus according to claim 2,
wherein the shift amount acquiring unit acquires the shift amount based on a specific portion of the subject in each of the radiological images.

10. The radiological image capturing and displaying apparatus according to claim 9,
wherein the specific portion is the outline of the subject.

11. The radiological image capturing and displaying apparatus according to claim 9,
wherein the subject is the breast and the specific portion is a nipple of the breast.

12. The radiological image capturing and displaying apparatus according to claim 2, further comprising:
a radiological image detector that has a rectangular shape and detects the radiological image,
wherein the shift amount acquiring unit acquires the shift amount based on the inclination of a side of the radiological image detector with respect to a straight line connecting the two focal positions.

13. The radiological image capturing and displaying apparatus according to claim 2, further comprising:
a radiological image compensating unit that performs a shift process on the two radiological images in the direction perpendicular to the parallax direction based on the shift amount acquired by the shift amount acquiring unit.

14. The radiological image capturing and displaying apparatus according to claim 3, further comprising:
a radiological image compensating unit that performs a shift process on the two radiological images in the direction perpendicular to the parallax direction based on the shift amount acquired by the shift amount acquiring unit.

15. The radiological image capturing and displaying apparatus according to claim 9, further comprising:
a radiological image compensating unit that performs a shift process on the two radiological images in the direction perpendicular to the parallax direction based on the shift amount acquired by the shift amount acquiring unit.

16. The radiological image capturing and displaying apparatus according to claim 2, further comprising:
a display control unit that controls the display unit to display the two radiological images which are shifted in the direction perpendicular to the parallax direction based on the shift amount acquired by the shift amount acquiring unit.

17. The radiological image capturing and displaying apparatus according to claim 3, further comprising:
a display control unit that controls the display unit to display the two radiological images which are shifted in the direction perpendicular to the parallax direction based on the shift amount acquired by the shift amount acquiring unit.

18. The radiological image capturing and displaying apparatus according to claim 9, further comprising:
a display control unit that controls the display unit to display the two radiological images which are shifted in the direction perpendicular to the parallax direction based on the shift amount acquired by the shift amount acquiring unit.

19. The radiological image capturing and displaying apparatus according to claim 2, further comprising:
a cassette detecting unit that detects the insertion of a removable cassette,
wherein the radiological image detector is provided in the removable cassette, and
when the cassette detecting unit detects the insertion of the cassette, the shift amount acquiring unit automatically acquires the shift amount, or information prompting the acquisition of the shift amount is notified.

20. The radiological image capturing and displaying apparatus according to claim 3, further comprising:
a cassette detecting unit that detects the insertion of a removable cassette,
wherein the radiological image detector is provided in the removable cassette, and
when the cassette detecting unit detects the insertion of the cassette, the shift amount acquiring unit automatically acquires the shift amount, or automatically notify the acquisition of the shift amount.
